(19) United States
(12) Patent Application Publication
(10) Pub. No.: US 2026/0083584 A1
(43) Pub. Date: Mar. 26, 2026

(54) FLUID COLLECTION ASSEMBLIES INCLUDING A FLUID IMPERMEABLE BARRIER HAVING A SUMP AND A BASE

(71) Applicant: PUREWICK CORPORATION, Covington, GA (US)

(72) Inventors: Adam Martin, Holly Springs, NC (US); Melody Mei Hee Kuroda, Durham, NC (US); Nicholas Jardine, Holly Springs, NC (US); Eric Alan Fallows, Apex, NC (US)

(21) Appl. No.: 19/337,217

(22) Filed: Sep. 23, 2025

Related U.S. Application Data
(63) Continuation of application No. 17/996,155, now Pat. No. 12,447,042.
(60) Provisional application No. 63/011,487, filed on Apr. 17, 2020.

(51) Int. Cl. A61F 5/455; A61F 5/44

(52) U.S. Cl. CPC A61F 5/455; A61F 5/4401; A61F 5/4404

(57) ABSTRACT

Embodiments disclosed herein are directed to fluid collection assemblies including a fluid impermeable barrier having a sump and a base. An example fluid collection assembly includes a fluid impermeable barrier. The fluid impermeable barrier includes a sump and a base. For example, the sump may protrude outwardly from the base. The sump defines an opening and a chamber. The fluid collection assembly may include at least one porous material disposed in the chamber that at least partially covers the opening, such as a fluid permeable membrane extending across at least a portion of the opening and a support disposed in the chamber. The fluid collection assembly may also include an adhesive layer disposed on the base that is configured to secure the rest of the fluid collection assembly to at least a portion of a pubic region of an individual. Further, the fluid collection assembly may include a fluid outlet.

### CROSS-REFERENCE TO RELATED APPLICATIONS

This application is a continuation application of U.S. patent application Ser. No. 17/996,155 filed on Oct. 13, 2022, which is a U.S. Nationalization of PCT International Application No. PCT/US2021/027425 filed on Apr. 15, 2021, which claims priority to U.S. Provisional Ser. No. 63/011,487 filed on Apr. 17, 2020, the disclosure of each of which is incorporated herein, in its entirety, by this reference.

### BACKGROUND

A person or animal may have limited or impaired mobility such that typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can be used to address some of these circumstances, such as incontinence. Unfortunately, urinary catheters can be uncomfortable, painful, and can lead to complications, such as infections. Additionally, bed pans, which are receptacles used for the toileting of bedridden individuals are sometimes used. However, bedpans can be prone to discomfort, spills, and other hygiene issues.

### SUMMARY

Embodiments disclosed herein are directed to fluid collection assemblies including a fluid impermeable barrier having a sump and a base. In an embodiment, a fluid collection assembly is disclosed. The fluid collection assembly includes a fluid impermeable barrier. The fluid impermeable barrier includes a base, a sump, and a fluid outlet. The sump defines a chamber and an opening configured to be positioned adjacent to a female urethral opening. The sump protrudes outwardly from the base. The fluid collection assembly also includes at least one porous material at least partially covering the opening and disposed in the chamber.

In an embodiment, a fluid collection system is disclosed. The fluid collection system includes a fluid collection assembly. The fluid collection assembly includes a fluid impermeable barrier. The fluid impermeable barrier includes a base, a sump, and a fluid outlet. The sump defines a chamber and an opening configured to be positioned adjacent to a female urethral opening. The sump protrudes outwardly from the base. The fluid collection assembly also includes at least one porous material at least partially covering the opening and disposed in the chamber. The fluid collection system also includes a fluid storage container in fluid communication with the fluid collection assembly and a vacuum device in fluid communication with the fluid collection assembly and the fluid storage container. The fluid collection assembly, the fluid storage container, and the vacuum device are configured to remove one or more bodily fluids from the chamber of the fluid collection assembly and deposit the bodily fluids in the fluid storage container when a suction force provided from the vacuum source is applied to the chamber of the fluid collection assembly.

In an embodiment, a method of using a fluid collection assembly is disclosed. The method includes securing a fluid collection assembly to at least a portion of a pubic region of an individual. The fluid collection assembly includes a base, a sump, and a fluid outlet. The sump defines a chamber and an opening configured to be positioned adjacent to a female urethral opening. The sump protrudes outwardly from the base. The fluid collection assembly also includes at least one porous material at least partially covering the opening and disposed in the chamber.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIGS. 1A to 1C are a front view, a back view, and an exploded front view of a fluid collection assembly, according to an embodiment.

FIG. 2A is an isometric view of a fluid collection assembly that is configured to have a fluid outlet thereof rotate, according to an embodiment.

FIG. 2B is an isometric view of the fluid collection assembly with the fluid outlet rotated, according to an embodiment.

FIGS. 3 and 4 are isometric views of different fluid collection assemblies exhibiting different shapes, according to different embodiments.

FIG. 5 is an isometric view of a fluid collection assembly, according to an embodiment.

FIG. 6 is an image of a fluid collection assembly disposed on a model of the female pubic region, according to an embodiment.

FIG. 7 is a block diagram of a system for fluid collection, according to an embodiment.

### DETAILED DESCRIPTION

Embodiments disclosed herein are directed to fluid collection assemblies including a fluid impermeable barrier having a sump and a base. An example fluid collection assembly includes a fluid impermeable barrier. The fluid impermeable barrier includes a sump and a base configured to be attached to an individual (e.g., user of the fluid collection assembly). The sump may protrude outwardly from the base. The sump defines an opening and a chamber. The fluid collection assembly may include at least one porous material disposed in the chamber that at least partially covers the opening, such as a fluid permeable membrane extending across at least a portion of the opening and a support disposed in the chamber. The fluid collection assembly may also include an adhesive layer disposed on the base that is configured to secure the rest of the fluid collection assembly to at least a portion of a pubic region of an individual. Further, the fluid collection assembly may include a fluid outlet.

The fluid collection assembly is configured to be disposed against the individual such that the opening is disposed adjacent to at least the urethral opening of the individual. The adhesive layer may be secured to one or more portions of the pubic region of the individual (e.g., at least one of including, adjacent to, or proximate to the vulva) to secure the fluid collection assembly to the individual and maintain the opening adjacent to the urethral opening. For example, the adhesive layer may be secured to one or more of the mons pubis, the inner thighs, the labia majora, the labia minora, the posterior commissure, the perineum, or other portions of the individual including, adjacent to, or proximate to the vulva. The adhesive layer may form a seal around a portion of the vulva of the individual to prevent bodily fluids from leaking from the fluid collection assembly.

After securing the fluid collection assembly, the individual may discharge, either controllably or uncontrollably, bodily fluids from the urethral opening (e.g., urine), vagina (e.g., blood), or other sources (e.g., sweat glands). The porous material may remove the bodily fluids from the individual and move the bodily fluids from the opening, through the chamber, and to the fluid outlet. The bodily fluids may be removed from the chamber via the fluid outlet by using at least one gravity (e.g., the fluid outlet is located at the gravimetric low point of the chamber) or a suction force provided by a vacuum which suctions the bodily fluids from the chamber.

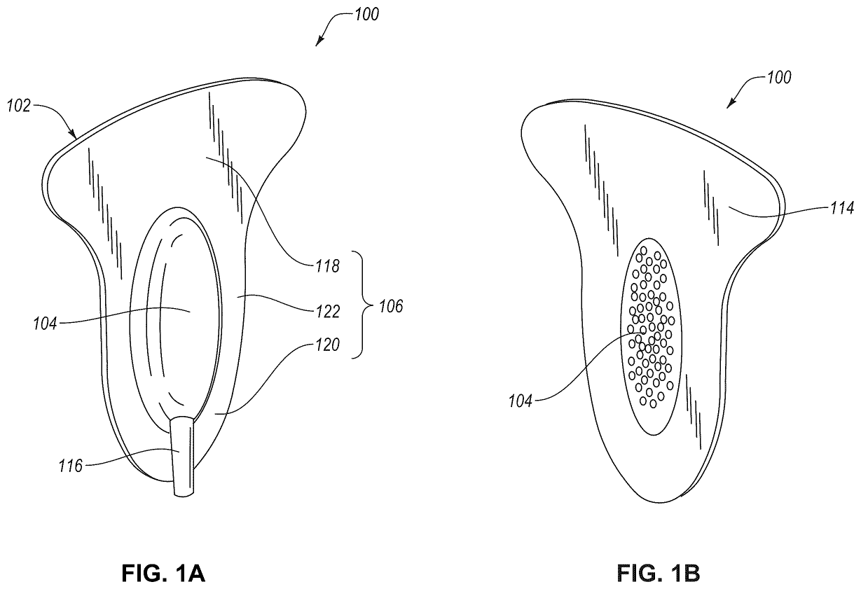

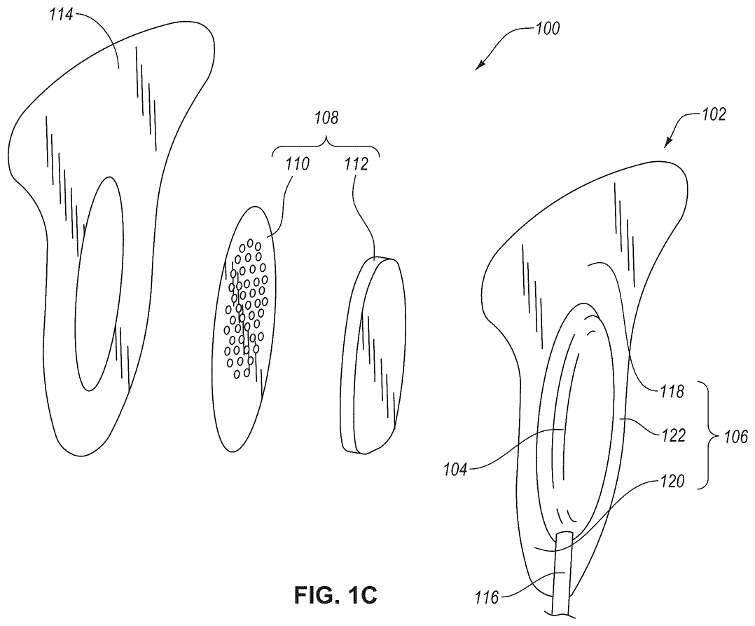

FIGS. 1A to 1C are a front view, a back view, and an exploded front view of a fluid collection assembly 100, according to an embodiment. The fluid collection assembly 100 includes a fluid impermeable barrier 102. The fluid impermeable barrier 102 includes a sump 104 and a base 106. The sump 104 extends outwardly from the base 106. The sump 104 defines an opening (covered by fluid permeable membrane 110) and a chamber (not shown, obscured). The fluid collection assembly 100 includes at least one porous material 108 that at least partially covers the opening and at least partially occupies the chamber. For example, the porous material 108 may include a fluid permeable membrane 110 that extends at least partially across the opening and a support 112 disposed in the chamber. The fluid collection assembly 100 also includes an adhesive layer 114 (e.g., a pressure sensitive adhesive) attached to a bottom surface of the base 106 (e.g., the surface of the base 106 that is adjacent to the skin on an individual during use and opposite the surface of the base 106 from which the sump 104 extends). The fluid collection assembly 100 may further include a fluid outlet 116.

The fluid impermeable barrier 102 is configured to receive and temporarily store the bodily fluids in the chamber of the sump 104. The fluid impermeable barrier 102 is also configured to substantially prevent the bodily fluids from leaking from the chamber. For example, as previously discussed, the sump 104 protrudes outwardly from the base 106 which allows the sump 104 to exhibit a volume in which the chamber and the opening may be formed. Meanwhile, the base 106 does not define or substantially does not define a volume that may receive and store the bodily fluids thereby allowing the sump 104 to receive and temporarily store most, if not all, of the bodily fluids. Further, the base 106 extends circumferentially about the sump 104 and extends outwardly from the sump 104 which allows the base 106, in conjunction with the adhesive layer 114, to form a seal around the sump 104 and attach the sump 104 to the individual.

The fluid impermeable barrier 102 may be formed of any suitable fluid imperious material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least a surface of the fluid impermeable barrier 102 that may contact the individual may be formed from a soft and/or smooth material (e.g., silicone), thereby reducing chaffing.

In an embodiment, the fluid impermeable barrier 102 may be formed from a flexible material, such as silicone, which allows the fluid impermeable barrier 102 to be bent into a shape that conforms the anatomy of the individual. For example, the anatomy of a individual at and near the vulva (i.e., mons pubis, the urethral opening, clitoris, labia minora, labium majora, vaginal opening, inner thighs, posterior commissure, perineal raphe, perineum, inner thighs, etc.) varies from individual to individual. As such, forming the fluid impermeable barrier 102 from a flexible material may allow the fluid impermeable barrier 102, such as the base 106, to be bent to exhibit a shape that corresponds to the anatomy of the individual which makes the fluid collection assembly 100 more comfortable to use and may inhibit the fluid collection assembly 100 from leaking. For example, the base 106 may be generally planar or may be curved to correspond to the shape of the anatomy of a hypothetical individual. Regardless if the base 106 is planar or curved, the base 106 may need to be shaped to correspond to a particular individual. However, it is noted that the base 106 may need to be shaped less if the base 106 exhibits a curved shape instead of a planar shape which, in turn, may make the fluid collection assembly 100 more comfortable to wear since such a base 106 may pull less on the skin of the individual than if the base 106 was planar.

As previously discussed, the sump 104 protrudes outwardly from the base 106 in a first direction. Protruding the sump 104 from the base 106 allows the sump 104 to exhibit a volume in which the chamber may be formed. The chamber may be sufficiently sized to have at least a portion of the porous material 108 disposed therein and to temporarily store the bodily fluids therein. The sump 104 also defines the opening which provides an ingress route for the bodily fluids to enter the chamber. In an embodiment, the opening may be an elongated hole that is configured to extend from above the urethral opening to below the vaginal opening. In such an embodiment, the opening may be able to allow bodily fluids discharged from both the urethral opening and the vaginal opening in the chamber. The size and shape of the sump 104 may allow the sump 104 to be comfortably positioned between the thighs of the individual using the fluid collection assembly 100. The sump 104, in conjunction with the porous material 108, may also exhibit sufficient rigidity such that any pressure applied from the thighs to the sump 104 is unlikely collapse the sump 104. Further, the sump 104, in conjunction with the porous material 108, may exhibit a r allow the sump 104 to spring back to

In an embodiment, as illustrated, the base 106 may include an upper portion 118 extending above the sump 104, a bottom portion 120 extending below the sump 104 (e.g., a side of the sump 104 opposite the upper portion 118), and two lateral portions 122 extending between the upper portion 118 and the bottom portion 120 that are on opposite sides of the sump 104. The upper portion 118 is configured to be adjacent to at least a portion of the mon pubis of the individual. The upper portion 118 may also be configured to be adjacent to other regions of the individual, such as a portion of the inner thighs. The bottom portion 120 is configured to be adjacent to at least one or more of the posterior commissure, at least a portion of the perineum (e.g., the perineal raphe), a portion of the inner thighs or a portion of the buttocks. The lateral portions 122 may be configured to be adjacent to at least at least one of the inner thighs or the labium majus. In an embodiment, the base 106 may be configured to not be adjacent to at least some of the more sensitive regions of the vulva of the individual, such as the labia minora and the clitoris, to avoid discomfort and pain when attaching and detaching the base 106 from such sensitive regions. However, it is noted that the base 106 maybe configured to be attached such sensitive regions of the vulva of the individual to at least one of form a better seal around the opening or minimize the regions of the vulva that may wetted by the bodily fluids. The base 106 may not be configured to be positioned adjacent to the urethral opening and, optionally, the vaginal opening so that the base 106 does not obstruct the flow of the bodily fluids from these orifices to the chamber.

The shape of the upper portion 118, the bottom portion 120, and the lateral portions 122 may be different since the upper portion 118, the bottom portion 120, and the lateral portions 122 may be configured to be adjacent to different portions of the individual. For example, the upper portion 118 may exhibit a maximum width that is generally greater than the maximum width of the base 106 at the bottom portion 120 or both lateral portions 122. The upper portion 118 may exhibit the greater maximum width because the mons pubis exhibits a wide width. However, the width of the upper portion 118 may decrease along at least a portion of a length therefrom from the maximum width to the lateral portions 122 since the width of the mons pubis generally decreases with increasing proximity to the urethral opening. In an embodiment, the upper portion 118 may exhibit a length measured from the sump 104 to an opposing edge thereof that is greater than a length of the bottom portion 120 measured from the sump 104 to an opposing edge thereof. The length of the upper portion 118 may be greater than the bottom portion 120 since, generally, the bottom portion 120 is configured to avoid the anus which restricts the length of the bottom portion 120 while the upper portion 118 does not have such a restriction. Further, increasing the length of the upper portion 120 increases the surface area of the individual to which the base 106 is attached which secures the fluid collection assembly 100 more securely to the individual and the upper portion 120 may support more of the weight of the fluid collection assembly 100 than the bottom portion 120. In an embodiment, the shape of the bottom portion 120 and the lateral portions 122 may be configured to avoid the inner thighs since, when the bottom portion 120 and/or the lateral portions 122 are attached to the inner thighs, movement of the legs of the individual may cause the base 106 to pull on the individual. In an embodiment, the shape of the bottom portion 120 and/or the lateral portions 122 may be configured to be attached to the inner thighs to at least one of avoid attaching the base 106 to the slightly more sensitive labium majora or to increase the surface area of the individual that the base 106 is attached (increasing the surface area that the base 106 is attached to may cause the base 106 to be more securely attached to the individual and decreases the likelihood that the fluid collection assembly 100 leaks). In an embodiment, as shown, the shape of the bottom portion 120 and/or the lateral portions 122 may conform to the shape of the sump 104 to ensure that there are no portions of the seal formed by the adhesive layer 114 that are thinner than other portions since the fluid collection assembly 100 is more likely to leak through such thin seals.

The fluid impermeable barrier 102 also forms and defines a fluid outlet 116. The fluid outlet 116 is an passageway formed in the fluid impermeable barrier 102 that is in fluid communication with the chamber. As such, the fluid outlet 116 may be used to remove bodily fluids from the chamber. The fluid outlet 116 is generally located at or near a gravimetric low point of the chamber to prevent pooling of the bodily fluids in the chamber. For example, the fluid outlet 116 may be in disposed or extend from a portion of the sump 104 that adjacent to the bottom portion 120 of the base 102 since such a portion of the sump 104 may be at or near the gravimetric low point of the chamber. The fluid outlet 116 may be sized to receive a conduit (not shown). The conduit may be disposed in the chamber or otherwise in fluid communication with the chamber via the fluid outlet 116. The fluid outlet 116 may be sized and shaped to form an at least substantially fluid tight seal against the conduit thereby substantially preventing the bodily fluids from escaping the chamber.

The fluid impermeable barrier 102 is configured to support one or more components of the fluid collection assembly 100. For example, the fluid impermeable barrier 102 may be configured to prevent the sump 104 from collapsing thereby allowing the porous material 108 to be disposed in the chamber and to at least partially cover the opening. The fluid impermeable barrier 102 may include one or more support pillars (not shown) to prevent at least one of the sump 104 from collapsing, the opening from closing, or the fluid outlet 116 from closing. In an embodiment, the support pillars may include increasing the thickness of the fluid impermeable barrier 102 in selected regions thereof to increase the strength of the fluid impermeable barrier 102. In an embodiment, the support pillars may include attaching a material, such a metal wires, to the rest of the fluid impermeable barrier 102 to increase the strength of the fluid impermeable barrier 102.

The fluid impermeable barrier 102 is configured to support the adhesive layer 114. The adhesive layer 114 may include any material that may secure the fluid impermeable barrier 102 to the individual. For example, the adhesive layer 114 may include a pressure adhesive. In an embodiment, as illustrated, the adhesive layer 114 may exhibit a shape that generally corresponds to the shape of the base 106. The adhesive layer 114 may also define a hole 124 to generally corresponds to at least one of the opening of the sump 104 or the portion of the porous material 108 that at least partially covers the opening. As such, the adhesive layer 114 does not obstruct the flow of bodily fluids through the opening and the porous material 108.

As previously discussed, the fluid collection assembly 100 includes porous material 108 disposed in the chamber. The porous material 108 may cover at least a portion (e.g., all) of the opening. The porous material 108 is exposed to the environment outside of the chamber through the opening. The permeable properties referred to herein may be wicking, capillary action, absorption, diffusion, or other similar properties or processes, and are referred to herein as “permeable” and/or “porous. ” The porous material 108 may also wick the bodily fluids generally towards an interior of the chamber, as discussed in more detail below. The porous material 108 may include one or more of a fluid permeable membrane 110 or a fluid permeable support 112.

In an embodiment, at least a portion of the porous material 108 may be a wicking material configured to wick any of the bodily fluids away from the opening, thereby preventing bodily fluids from escaping the chamber. The wicking material may not include absorption of the bodily fluids into the wicking material. Put another way, substantially no absorption of the bodily fluids into the wicking material may take place after the wicking material is exposed to the bodily fluids. While no absorption is desired, the term “substantially no absorption” may allow for nominal amounts of absorption of the bodily fluids into the wicking material (e.g., absorbency), such as about 30 wt% of the dry weight of the wicking material, about 20 wt%, about 10 wt%, about 7 wt%, about 5 wt%, about 3 wt%, about 2 wt%, about 1 wt%, or about 0.5 wt% of the dry weight of the wicking material.

The fluid collection assembly 100 may include the fluid permeable membrane 110 disposed in the chamber. The fluid permeable membrane 110 may cover at least a portion (e.g., all) of the opening. The fluid permeable membrane 110 may be composed to pull/push the bodily fluids away from the opening, thereby promoting fluid flow into the sump 104, prevent fluid remaining on the vulva of the individual, and preventing the bodily fluids from escaping the chamber.

The fluid permeable membrane 110 may include any material that may be permeable to the bodily fluids. For example, the fluid permeable membrane 110 may include fabric, such as a gauze (e.g., a silk, linen, or cotton gauze), another soft fabric, or another smooth fabric. Forming the fluid permeable membrane 110 from gauze, soft fabric, and/or smooth fabric may reduce chaffing caused by the fluid collection assembly 100 and makes wearing the fluid collection assembly more comfortable. In an embodiment, the fluid permeable membrane 110 may define a plurality of perforations that are larger than an inherent porosity of the fluid permeable membrane 110. The plurality of perforations formed in the fluid permeable membrane 110 may extend completely through the fluid permeable membrane 110. The plurality of perforations may increase the rate at which bodily fluids enter the chamber. In an embodiment, the fluid permeable membrane 110 may be continuous (e.g., does not define perforations therein) and only includes the inherent porosity thereof.

The fluid collection assembly 100 may include the fluid permeable support 112 disposed in the chamber. The fluid permeable support 112 is configured to support the fluid permeable membrane 110 and maintain the shape of the sump 104 since the fluid impermeable barrier 102 and the fluid permeable membrane 110 may be formed from a relatively foldable, flimsy, or otherwise easily deformable material. For example, the fluid permeable support 112 may be positioned such that the fluid permeable membrane 110 is disposed between the fluid permeable support 112 and the fluid impermeable barrier 102. As such, the fluid permeable support 112 may support and maintain the position of the fluid permeable membrane 110 and the shape of the sump 104. The fluid permeable support 112 may include any material that may be permeable to the bodily fluids, such as any of the fluid permeable membrane materials disclosed herein above. For example, the fluid permeable membrane material(s) may be utilized in a more dense or rigid form than in the fluid permeable membrane 110 when used as the fluid permeable support 112. The fluid permeable support 112 may be formed from any fluid porous material that is less deformable than the fluid permeable membrane 110. For example, the fluid permeable support 112 may include a porous polymer (e.g., nylon, polyester, polyurethane, polyethylene, polypropylene, etc.) structure (e.g., spun fibers) or a foam (e.g., an open cell foam). In a particular example, the fluid permeable support 112 may include spun fibers, such as spun nylon fibers. In some examples, the fluid permeable support 112 may be formed from a natural material, such as cotton, wool, silk, or combinations thereof. In such examples, the material may have a coating to prevent or limit absorption of the bodily fluids into the material, such as a water repellent coating. In some examples, the fluid permeable support 112 may be formed from fabric, felt, gauze, or combinations thereof.

In some examples, the fluid permeable membrane 110 may be optional. For example, the porous material 108 may include only the fluid permeable support 112. In some examples, the fluid permeable support 112 may be optionally omitted from the fluid collection assembly 100. For example, the porous material 108 may only include the fluid permeable membrane 110.

In an embodiment, the fluid permeable membrane 110 and/or the fluid permeable support 112 are wicking materials. In such an embodiment, the fluid permeable support 112 may have a greater ability to wick the bodily fluids than the fluid permeable membrane 110, such as to move the bodily fluids inwardly from the outer surface of the fluid collection assembly 100. In some examples, the wicking ability of the fluid permeable support 112 and the fluid permeable membrane 110 may be substantially the same. In an embodiment, the fluid permeable membrane 110 and/or the fluid permeable support 112 are non-wicking materials (e.g., absorbent materials).

In an embodiment, not shown, the fluid permeable membrane 110 and the fluid permeable support 112 may at least substantially completely fill the portions of the chamber that are not occupied by the conduit. In an embodiment, as previously discussed, the fluid permeable membrane 110 and the fluid permeable support 112 may not substantially completely fill the portions of the chamber that are not occupied by the conduit. In such an embodiment, the fluid collection assembly 100 includes the fluid reservoir (not shown) disposed in the chamber.

The fluid reservoir is a substantially unoccupied portion of the chamber. The fluid reservoir may be defined between the fluid impermeable barrier 102 and one or both of the fluid permeable membrane 110 and fluid permeable support 112. The bodily fluids that are in the chamber may flow through the fluid permeable membrane 110 and/or fluid permeable support 112 to the fluid reservoir. The fluid reservoir may retain of the bodily fluids therein. The bodily fluids that are in the chamber may flow through the fluid permeable membrane 110 and/or fluid permeable support 112 and, optionally, to the fluid reservoir. The fluid impermeable barrier 102 may retain the bodily fluids in the fluid reservoir. The fluid reservoir may be located in a portion of the chamber that is designed to be located in a gravimetrically low point of the fluid collection assembly when the device is worn.

As previously discussed, the fluid collection assembly 100 may include a conduit (not shown). The conduit may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit may include silicon or latex. In some examples, the conduit may include one or more portions that are resilient, such as to by having one or more of a diameter or wall thickness that allows the conduit to be flexible.

In an example, the conduit is configured to be at least insertable into the chamber. In such an example, the conduit may include one or more markers (not shown) on an exterior thereof that are located to facilitate insertion of the conduit into the chamber. For example, the conduit may include one or more markings thereon that are configured to prevent over or under insertion of the conduit, such as when the conduit defines an inlet that is configured to be disposed in or adjacent to the reservoir. In another example, the conduit may include one or more markings thereon that are configured to facilitate correct rotation of the conduit relative to the chamber. The one or more markings may include a line, a dot, a sticker, or any other suitable marking.

As described in more detail below, the conduit is configured to be coupled to, and at least partially extend between, one or more of the fluid storage container (not shown) and the vacuum source (not shown). In an example, the conduit is configured to be directly connected to the vacuum source (not shown). In such an example, the conduit may extend from the fluid impermeable barrier 102 by at least one foot, at least two feet, at least three feet, or at least six feet. In another example, the conduit is configured to be indirectly connected to at least one of the fluid storage container (not shown) and the vacuum source (not shown). In some examples, the conduit is secured to a wearer's skin with a catheter securement device, such as a STATLOCK® catheter securement device available from C. R. Bard, Inc., including but not limited to those disclosed in U.S. Pat. Nos. 6,117,163; 6,123,398; and 8,211,063, the disclosures of which are all incorporated herein by reference in their entirety.

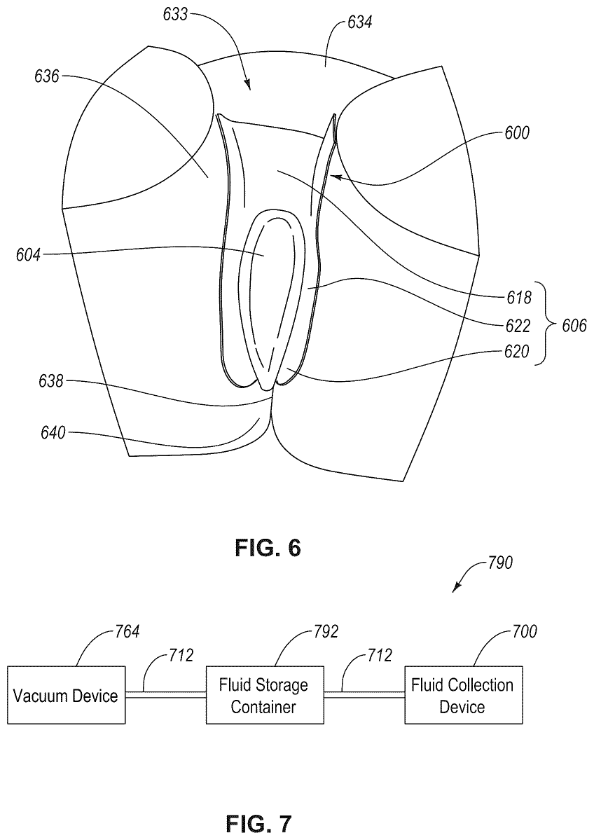

The inlet and the outlet of the conduit are configured to fluidly couple (e.g., directly or indirectly) the vacuum source (not shown) to the chamber (e.g., the reservoir). As the vacuum source (FIG. 6) applies a vacuum/suction in the conduit, the bodily fluids in the chamber may be drawn into the conduit and out of the fluid collection assembly. In some examples, the conduit may be frosted or opaque (e.g., black) to obscure visibility of the bodily fluids therein.

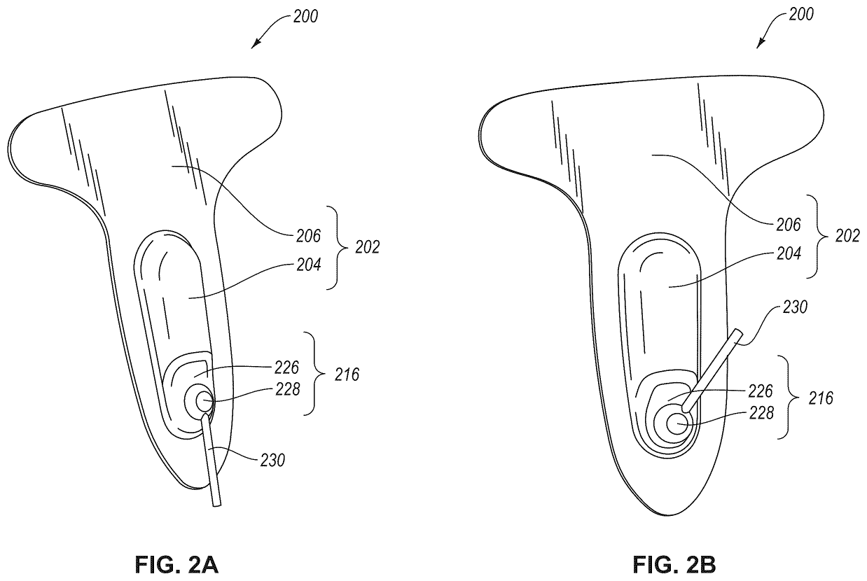

FIG. 2A is an isometric view of a fluid collection assembly 200 that is configured to have a fluid outlet 216 thereof rotate, according to an embodiment. FIG. 2B is an isometric view of the fluid collection assembly 200 with the fluid outlet 216 rotated, according to an embodiment. Except as otherwise disclosed herein, the fluid collection assembly 200 is the same or substantially similar to any of the fluid collection assemblies disclosed herein. For example, the fluid collection assembly 200 may include a fluid impermeable barrier 202 including a sump 204 and a base 206. The sump 204 may define a chamber (not shown, obscured) and an outlet (not shown, obscured). The fluid collection assembly 200 may include at least one porous material (not shown, obscured) and an adhesive layer (not shown, obscured).

The fluid collection assembly 200 includes a fluid outlet 216. The fluid outlet 216 is generally located at or near the bottom of the sump 204 (e.g., the portion of the sump 204 closest to the anus during use) since such a location is typically at or near the gravimetric low point of the sump 204 during use. The fluid outlet 216 includes a stationary element 226 and a rotating element 228. The stationary element 226 remains stationary relative to the sump 204 and the rotating element 228 is configured to rotate relative to the stationary element 226. As such, the stationary element 226 and the rotating element 228 may form a bearing element therebetween. The stationary element 226 and the rotating element 228 may be configured to fit together snugly to inhibit fluid leaking thereby. The rotating element 228 may be configured to receive a conduit 230, as previously discussed herein.

In an embodiment, the stationary element 226 may be formed from the same material as the rest of the fluid impermeable barrier 202. In an embodiment, the stationary element 226 may be formed from a material that is different than the rest of the fluid impermeable barrier 202. For example, the stationary element 226 may be formed from a more rigid material (e.g., stiffer and/or stronger material) than the rest of the fluid impermeable barrier 202 to prevent the rotating element 228 from being dislodged therefrom. In such an example, the rotating element 228 may also be formed from a material that is more rigid that the rest of the fluid impermeable barrier 202. In an embodiment, the material of the stationary element 226 is selected to exhibit a lower coefficient of friction (e.g., kinetic and/or static coefficient of friction) with the rotating element 228 than the rest of the fluid impermeable barrier 202.

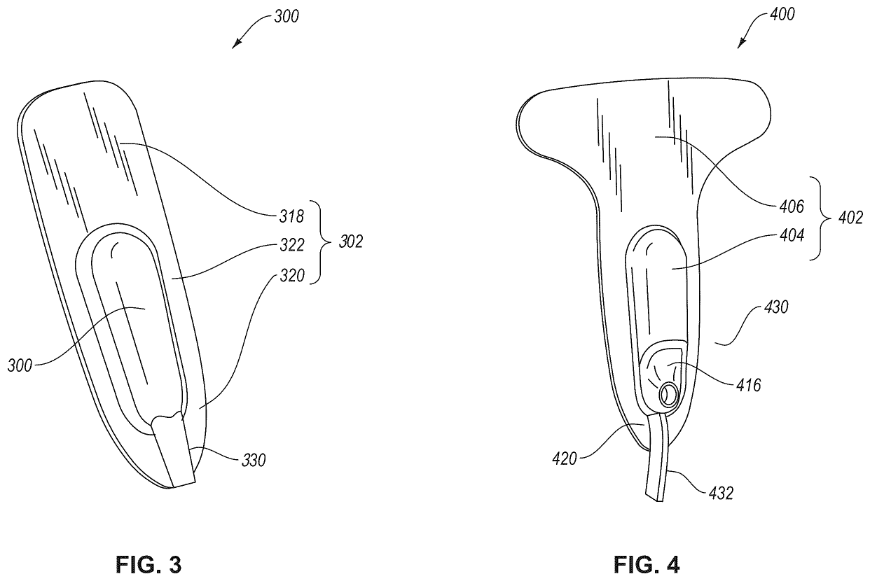

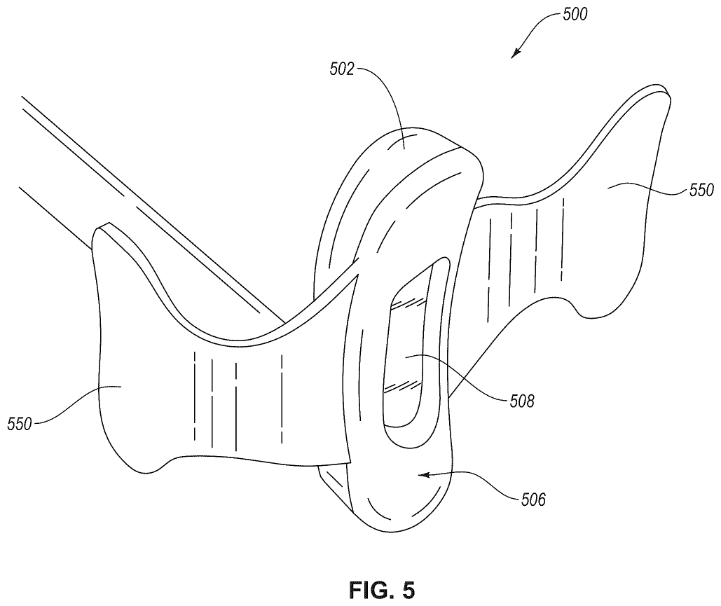

The fluid collection assemblies disclosed herein may exhibit shapes other than the shapes illustrated in FIGS. 1A-2B. For example, FIGS. 3 and 4 are isometric views of different fluid collection assemblies exhibiting different shapes, according to different embodiments. Except as otherwise disclosed herein, the fluid collection assemblies illustrated in FIGS. 3 and 4 are the same or substantially similar to any of the fluid collection assemblies disclosed herein. For example, the fluid collection assemblies may include a fluid impermeable barrier having a sump and a base. The fluid collection assemblies may also include at least one porous material, an adhesive layer, a fluid outlet, a conduit, or any other component disclosed herein.

Referring to FIG. 3, the fluid collection assembly 300 includes a base 306. The base includes an upper portion 318, a bottom portion 320, and two lateral portions 322. The upper portion 318 exhibits a maximum width that is substantially the same (e.g., ±30%, ±20%, ±10%, ±5%, or the same) as the maximum width of the bottom portion 320 or the maximum width of the two lateral portions 322 and the sump 304. For example, the upper portion 318 may not need to exhibit a maximum width that is significantly greater than the maximum width of the bottom portion 320 or the maximum width of the two lateral portions 322 and the sump 304 to secure the fluid collection assembly 300 to the individual. Further, reducing the maximum width of the upper portion 318 may minimize the pain and discomfort caused by detaching the fluid collection assembly 300 from the individual.

Referring to FIG. 4, the fluid collection assembly 400 includes a sump 404 and a base 406. The fluid collection assembly 400 includes a tail 432 attached to or integrally formed with at least one of the sump 404, a bottom portion 420 of the base 406, or the fluid outlet 416. The tail 432 extends away from the sump 404. The tail 432 is configured to fit between the gluteal cleft of the individual to better secure the fluid collection assembly 400 to the individual.

FIG. 5 is an isometric view of a fluid collection assembly 500, according to an embodiment. Except as otherwise disclosed herein, the fluid collection assembly 500 is the same or substantially similar to any of the fluid collection assemblies disclosed herein. For example, the fluid collection assembly 500 may include a fluid impermeable barrier 502 that includes a sump 504 and a base 506. The fluid collection assembly 500 may also include at least one porous material 508 disposed in the sump 504.

The base 506 may include two lateral wings 550 extending from the sump 504. The lateral wings 550 may be configured to be positioned adjacent to at least one of the labia minora, the labia majora, or the inner thighs. In the illustrated embodiment, the lateral wings 550 exhibit a generally hour-glass like shape. However, it is noted that the lateral wings 550 may exhibit other shapes, such as a generally rectangular shape (e.g., generally square shape), a generally triangular shape, or a generally trapezoidal shape.

In an embodiment, as illustrated, the base 506 may be integrally formed with the adhesive layer of the fluid collection assembly 500. In other words, the base 506 directly secures the fluid collection assembly 500 to the individual. In such an embodiment, the base 506 may include an adhesive or a tacky surface. The tacky surface may push the fluid collection assembly 500 towards the urethral opening of the individual. When the base 506 is integrally formed with the adhesive layer, the base 506 may correspond to and cover one or more surfaces of the sump 504. In an embodiment, the fluid collection assembly 500 may include an adhesive layer (not shown) that is distinct from and attached to the base 506.

FIG. 6 is an isometric view of a fluid collection assembly 600 disposed on a portion of the female pubic region 633, according to an embodiment. As illustrated, the fluid collection assembly 600 is substantially similar to the fluid collection assembly 100 illustrated in FIGS. 1A-1C. However, it is noted that the fluid collection assembly 600 may be the same or substantially similar to any of the fluid collection assemblies disclosed herein. The fluid collection assembly 600 includes a sump 604 and a base 606. The base 606 includes an upper portion 618 that is positioned adjacent to and attached to (via the adhesive layer, obscured) to the mons pubis 634 and the inner thighs 636 of the model 633. The base 606 also includes a bottom portion 620 that is positioned adjacent to and attached to (via the adhesive layer) to the perineum 638 and the buttocks 640 of the female pubic region 633. The bottom portion 620 may also be positioned adjacent to and attached to (via the adhesive layer) to a portion of the inner thighs 636 of the female pubic region 633. The base 606 also includes two lateral portions 622 that are positioned adjacent to and attached to (via the adhesive layer) to the inner thighs 636 of the female pubic region 633. It is noted that the base 606 may also be attached to portions of the vulva (not shown, obscured) of the female pubic region 633, such as the labium majora. FIG. 6 illustrates that the fluid collection assemblies disclosed herein may conform to the female anatomy.

The fluid collection assemblies disclosed herein may are discussed as being used to collect bodily fluids from a female. However, it is noted that the principles discussed herein may be used to collect bodily fluids from a male (e.g., from a penis). In an example, any of the fluid collection assemblies disclosed herein may be used to collect bodily fluids from a buried penis. In such an example, the openings of the fluid collection assemblies may be configured to be positioned adjacent to the buried penis and the base may be configured to be attached to or around (via the adhesive layer) to the testicles. In an example, the opening of a fluid collection assembly that is substantially similar to any of the fluid collection assemblies disclosed herein may be configured to receive the penis into the chamber. In such an example, the base may be configured to be attached to or around (via the adhesive layer) to the testicles.

FIG. 7 is a block diagram of a system 790 for fluid collection, according to an embodiment. The system 790 includes a fluid collection assembly 700, a fluid storage container 792, and a vacuum source 764. The fluid collection assembly 700, the fluid storage container 792, and the vacuum source 764 may be fluidly coupled to each other via one or more conduits 712. For example, fluid collection assembly 700 may be operably coupled to one or more of the fluid storage container 792 or the vacuum source 764 via the conduit 712. However, as previously discussed, the vacuum source 764 and the fluid storage container 792 may be integrally formed together. The bodily fluids collected in the fluid collection assembly 700 may be removed from the fluid collection assembly 700 via the conduit 712 which protrudes into the fluid collection assembly 700. For example, an inlet of the conduit 712 may extend into the fluid collection assembly 700, such as to a fluid reservoir therein. The outlet of the conduit 712 may extend into the fluid collection assembly 700 or the vacuum source 764. Suction force may be introduced into the chamber of the fluid collection assembly 700 via the inlet of the conduit 712 responsive to suction (e.g., vacuum) force applied at the outlet of the conduit 712.

The suction force may be applied to the outlet of the conduit 712 by the vacuum source 764 either directly or indirectly. The suction force may be applied indirectly via the fluid storage container 792. For example, the outlet of the conduit 712 may be disposed within the fluid storage container 792 and an additional conduit 712 may extend from the fluid storage container 792 to the vacuum source 764. Accordingly, the vacuum source 764 may apply suction to the fluid collection assembly 700 via the fluid storage container 792. The suction force may be applied directly via the vacuum source 764. For example, the outlet of the conduit 712 may be disposed within the vacuum source 764. An additional conduit 712 may extend from the vacuum source 764 to a point outside of the fluid collection assembly 700, such as to the fluid storage container 792. In such examples, the vacuum source 764 may be disposed between the fluid collection assembly 700 and the fluid storage container 792.

The fluid collection assembly 700 may be similar or identical to any of the fluid collection assemblies disclosed herein in one or more aspects. The fluid collection assembly 700 may be shaped and sized to be positioned adjacent to a female urethra or have a male urethra positioned therethrough (e.g., receive a penis therein). For example, the fluid collection assembly 700 may include a fluid impermeable barrier at least partially defining a chamber (e.g., interior region) of the fluid collection assembly 700. The fluid impermeable barrier also defines an opening extending therethrough from the external environment. The opening may be positioned adjacent to a female urethra or have a male urethra positioned therethrough. The fluid collection assembly 700 may include a fluid permeable membrane disposed within the fluid impermeable barrier. The fluid collection assembly 700 may include at least one porous material disposed in the chamber such as one or more of a fluid permeable support and a fluid permeable membrane.

The fluid storage container 792 is sized and shaped to retain the bodily fluids therein. The fluid storage container 792 may include a bag (e.g., drainage bag), a bottle or cup (e.g., collection jar), or any other enclosed container for storing bodily fluids such as urine. In some examples, the conduit 712 may extend from the fluid collection assembly 700 and attach to the fluid storage container 792 at a first point therein. An additional conduit 712 may attach to the fluid storage container 792 at a second point thereon and may extend and attach to the vacuum source 764. Accordingly, a vacuum (e.g., suction) may be drawn through fluid collection assembly 700 via the fluid storage container 792. The bodily fluids, such as urine, may be drained from the fluid collection assembly 700 using the vacuum source 764.

The vacuum source 764 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 764 may provide a vacuum or suction to remove the bodily fluids from the fluid collection assembly 700. In some examples, the vacuum source 764 may be powered by one or more of a power cord (e.g., connected to a power socket), one or more batteries, or even manual power (e.g., a hand operated vacuum pump). In some examples, the vacuum source 764 may be sized and shaped to fit outside of, on, or within the fluid collection assembly 700. For example, the vacuum source 764 may include one or more miniaturized pumps or one or more micro pumps. The vacuum sources 764 disclosed herein may include one or more of a switch, a button, a plug, a remote, or any other device suitable to activate the vacuum source 764.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or +2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

What is claimed is:

1. A fluid collection assembly, comprising:
a base;
a sump defining a chamber and an opening, the sump protruding outwardly from the base;
a fluid outlet, wherein at least a portion of the fluid outlet is rotatable relative to at least one of the base or the sump; and
at least one porous material at least partially covering the opening and disposed in the chamber.

2. The fluid collection assembly of claim 1, wherein the base includes two wings extending outwardly from the sump.

3. The fluid collection assembly of any one of claims 1, wherein the base includes an upper portion, a bottom portion, and at least one lateral portion extending between the upper portion and the bottom portion.

4. The fluid collection assembly of claim 3, wherein a maximum width of the upper portion is greater than a maximum width of the bottom portion.

5. The fluid collection assembly of claim 3, wherein the bottom portion and the at least one lateral portion exhibit a shape that generally corresponds to the shape of the sump.

6. The fluid collection assembly of claim 3, wherein at least one of the upper portion, the bottom portion, or the at least one lateral portion extend outwardly from the sump.

7. The fluid collection assembly of claim 3, wherein the base exhibits single piece construction with the sump.

8. The fluid collection assembly of claim 1, wherein the fluid outlet extends from a portion of the sump that is closest to an anus during use.

9. The fluid collection assembly of claim 1, wherein fluid outlet includes a stationary element and a rotating element attached to the stationary element, the stationary element configured to remain stationary relative to the sump and the rotating element configured to rotate relative to the stationary element.

10. The fluid collection assembly of claim 9, wherein the stationary element includes at least one material that is different than the sump.

11. The fluid collection assembly of claim 10, wherein the stationary element exhibits a rigidity that is greater than the sump.

12. The fluid collection assembly of claim 9, wherein the stationary element is attached to the sump.

13. The fluid collection assembly of claim 9, wherein the rotating element is configured to be coupled to a conduit.

14. The fluid collection assembly of claim 9, wherein the sump defines a depression and the fluid outlet is positioned within the depression.

15. The fluid collection assembly of claim 1, wherein the at least one porous material includes a fluid permeable membrane and a fluid permeable support, wherein the fluid permeable membrane is disposed on and supported by the fluid permeable support such that the fluid permeable membrane extends across the opening.

16. The fluid collection assembly of claim 15, wherein the fluid permeable membrane defines a plurality of perforations therein, wherein the plurality of perforations are larger than a porosity of the fluid permeable membrane.

17. The fluid collection assembly of claim 1, further comprising an adhesive layer disposed on a first surface of the base that is configured to be positioned adjacent to an individual.

18. The fluid collection assembly of claim 1, further comprising a tail extending from the base, the sump, or the fluid outlet, the tail configured to fit between the gluteal cleft of an individual.

19. A fluid collection system, comprising:
a fluid collection assembly including:
a base;
a sump defining a chamber and an opening, the sump protruding outwardly from the base;
a fluid outlet, wherein at least a portion of the fluid outlet is rotatable relative to at least one of the base or the sump; and
at least one porous material at least partially covering the opening and disposed in the chamber;
a fluid storage container in fluid communication with the fluid collection assembly; and
a vacuum device in fluid communication with the fluid collection assembly and the fluid storage container;
wherein the fluid collection assembly, the fluid storage container, and the vacuum device are configured to remove one or more bodily fluids from the chamber of the fluid collection assembly and deposit the bodily fluids in the fluid storage container when a suction force provided from the vacuum source is applied to the chamber of the fluid collection assembly.

20. A method of using a fluid collection assembly, the method comprising:
securing a fluid collection assembly to at least a portion of a pubic region of an individual, the fluid collection assembly including:
a base;
a sump defining a chamber and an opening, the sump protruding outwardly from the base;
a fluid outlet rotatable relative to at least one of the base or the sump; and
at least one porous material at least partially covering the opening and disposed in the chamber.

21. The method of claim 20, further comprising rotating at least a portion of the fluid outlet relative to at least one of the base or the sump.
